(19) United States
(12) Patent Application Publication
(10) Pub. No.: US 2023/0397890 A1
(43) Pub. Date: Dec. 14, 2023

(54) FATIGUE LEVEL ESTIMATION APPARATUS, FATIGUE LEVEL ESTIMATION METHOD, AND COMPUTER-READABLE RECORDING MEDIUM

(71) Applicant: NEC Corporation, Minato-ku, Tokyo (JP)

(72) Inventors: Jingwen LU, Tokyo (JP); Tasuku Kitade, Tokyo (JP); Masanori Tsujikawa, Tokyo (JP)

(73) Assignee: NEC Corporation, Minato-ku, Tokyo (JP)

(21) Appl. No.: 18/033,659

(22) PCT Filed: Nov. 10, 2020
(86) PCT No.: PCT/JP2020/041938

§ 371 Date: Apr. 25, 2023

(51) Int. Cl. A61B 5/00; A61B 5/024

(52) U.S. Cl. CPC A61B 5/7275; A61B 5/4809; A61B 5/4812; A61B 5/024; A61B 5/7264

(57) ABSTRACT

A fatigue level estimation apparatus includes: a biological data extraction unit that extracts biological data obtained when a subject is in a specific activity state, from biological data obtained from the subject; a feature value calculation unit that calculates a feature value of the biological data, based on the extracted biological data obtained when the subject is in a specific activity state; and a fatigue level estimation unit that estimates a fatigue level indicating a level of fatigue of the subject, based on the calculated feature value.

### TECHNICAL FIELD

The present invention relates to a fatigue level estimation apparatus and a fatigue level estimation method for estimating the fatigue level of a person from biological data, and further relates to a computer-readable recording medium on which a program for realizing the apparatus and method is recorded.

### BACKGROUND ART

Recent improvements in sensor technology have made it easier to obtain human biological data. Accordingly, a fatigue level indicating the degree to which a person is fatigued is estimated by obtaining an electrocardiographic signal as biological data, for example. Fatigue level estimation is important in terms of improving productivity of companies and the like.

In view of this, Patent Document 1 discloses an apparatus for estimating the fatigue level. The apparatus disclosed in Patent Document 1 obtains an electrocardiographic signal and a photoelectronic pulse wave signal from a subject as the biological data and calculates a pulse wave transfer time from the time difference between peaks of the two signals. The apparatus disclosed in Patent Document 1 estimates the current fatigue level of the subject by applying the calculated pulse wave transfer time to the pre-obtained relative relationship between the pulse wave transfer time and the fatigue level.

### LIST OF RELATED ART DOCUMENTS

### Patent Document

Patent Document 1: International Patent Laid-Open Publication No. 2014/208289

### SUMMARY OF INVENTION

### Problems to be Solved by the Invention

Incidentally, biological data such as an electrocardiographic signal fluctuates depending on the state of the autonomous nerve system as well as the fatigue level. Accordingly, in order to accurately estimate the fatigue level, biological data needs to be obtained in a state where the autonomous nerve system is stable. The biological data also fluctuates depending on the state of activity of the person, in addition to the fatigue level and the state of the autonomous nerve system.

Accordingly, with the apparatus disclosed in Patent Document 1, it is difficult to accurately estimate the fatigue level from the biological data. In order to accurately estimate the fatigue level, it is necessary to obtain highly reproducible biological data, that is, biological data that does not include any component that causes the fatigue level to fluctuate.

An example object of the invention is to provide a fatigue level estimation apparatus, a fatigue level estimation method, and a computer-readable recording medium capable of solving the above problem and estimating the fatigue level by obtaining biological data that does not include any component that causes the fatigue level to fluctuate.

### Means for Solving the Problems

In order to achieve the above-described object, a fatigue level estimation apparatus includes:
a biological data extraction unit that extracts biological data obtained when a subject is in a specific activity state, from biological data obtained from the subject;
a feature value calculation unit that calculates a feature value of the biological data, based on the extracted biological data obtained when the subject is in a specific activity state; and
a fatigue level estimation unit that estimates a fatigue level indicating a level of fatigue of the subject, based on the calculated feature value.

In addition, in order to achieve the above-described object, a fatigue level estimation method includes:
a biological data extraction step of extracting biological data obtained when a subject is in a specific activity state, from biological data obtained from a subject;
a feature value calculation step of calculating a feature value of the biological data, based on the extracted biological data obtained when the subject is in a specific activity state; and
a fatigue level estimation step of estimating a fatigue level indicating a level of fatigue of the subject, based on the calculated feature value.

Furthermore, in order to achieve the above-described object, a computer readable recording medium according to an example aspect of the invention is a computer readable recording medium that includes recorded thereon a program,
the program including instructions that cause a computer to carry out:
a biological data extraction step of extracting biological data obtained when a subject is in a specific activity state, from biological data obtained from a subject;
a feature value calculation step of calculating a feature value of the biological data, based on the extracted biological data obtained when the subject is in a specific activity state; and
a fatigue level estimation step of estimating a fatigue level indicating a level of fatigue of the subject, based on the calculated feature value.

### Advantageous Effects of the Invention

As described above, according to the invention, it is possible to estimate the fatigue level by obtaining biological data that does not include any component that causes the fatigue level to fluctuate.

### BRIEF DESCRIPTION OF THE DRAWINGS

FIG. 1 is a block diagram showing a schematic configuration of the fatigue level estimation apparatus according to the first example embodiment.

FIG. 2 is a block diagram specifically showing the configuration of the fatigue level estimation apparatus according to the first example embodiment.

FIG. 3 is a diagram for illustrating the calculation processing of the feature value of the first example embodiment. FIG. 3(a) shows an example of original RRI data, FIG. 3(b) shows resampled RRI data, and FIG. 3(c) shows RRI data that has been subjected to frequency transformation.

FIG. 4 is a flowchart showing the operation of the fatigue level estimation apparatus of the first example embodiment.

FIG. 5 is a block diagram showing the configuration of the fatigue level estimation apparatus according to the second example embodiment.

FIG. 6 is a flowchart showing the operation of the fatigue level estimation apparatus according to the second example embodiment.

FIG. 7 is a flowchart showing operations in processing for detecting the activity state performed by the fatigue level estimation apparatus in the second example embodiment.

FIG. 8 is a flowchart showing another operation in detection processing of the activity state performed by the fatigue level estimation apparatus in the second example embodiment.

FIG. 9 is a block diagram illustrating an example of a computer that realizes the fatigue level estimation apparatus according to the first and second example embodiment.

### EXAMPLE EMBODIMENT

### First Example Embodiment

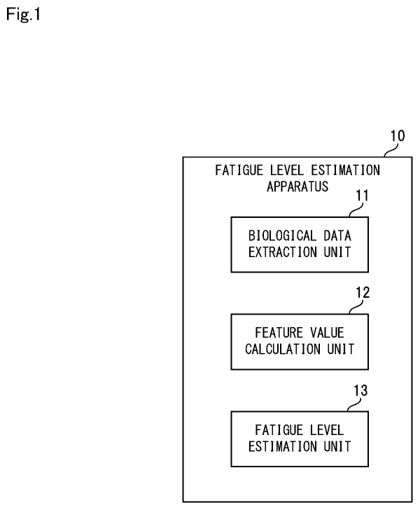

Hereinafter, a fatigue level estimation apparatus, a fatigue level estimation method, and a program according to a first example embodiment will be described with reference to FIGS. 1 to 4.

[Apparatus Configuration]

First, the overall configuration of a fatigue level estimation apparatus according to the first example embodiment will be described using FIG. 1. FIG. 1 is a block diagram showing a schematic configuration of the fatigue level estimation apparatus according to the first example embodiment.

A fatigue level estimation apparatus 10 according to the first example embodiment shown in FIG. 1 is an apparatus for estimating the fatigue level of a subject from biological data. Here, “fatigue” includes both physical fatigue and mental fatigue. The fatigue level estimation apparatus 10 estimates the level of both physical fatigue and mental fatigue. As shown in FIG. 1, the fatigue level estimation apparatus 10 includes a biological data extraction unit 11, a feature value calculation unit 12, and a fatigue level estimation unit 13.

The biological data extraction unit 11 extracts biological data obtained when the subject is in a specific activity state from the biological data obtained from the subject. The feature value calculation unit 12 calculates the feature value of the biological data based on the biological data obtained when the subject is in a specific activity state, the biological data being extracted by the biological data extraction unit 11. The fatigue level estimation unit 13 estimates the fatigue level indicating the level of fatigue of the subject based on the feature value calculated by the feature value calculation unit 12.

In this manner, in the first example embodiment, the fatigue level is estimated using only the biological data obtained when the subject is in a specific activity state. That is, according to the first example embodiment, biological data that does not include any component that causes the fatigue level to fluctuate can be obtained to estimate the fatigue level.

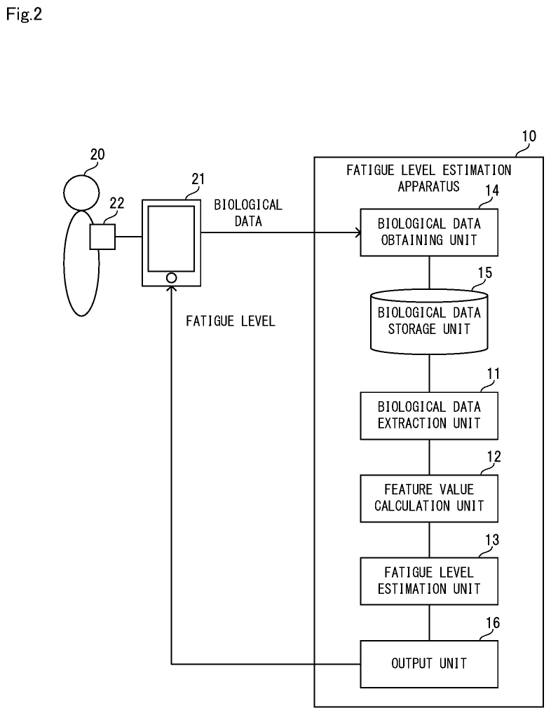

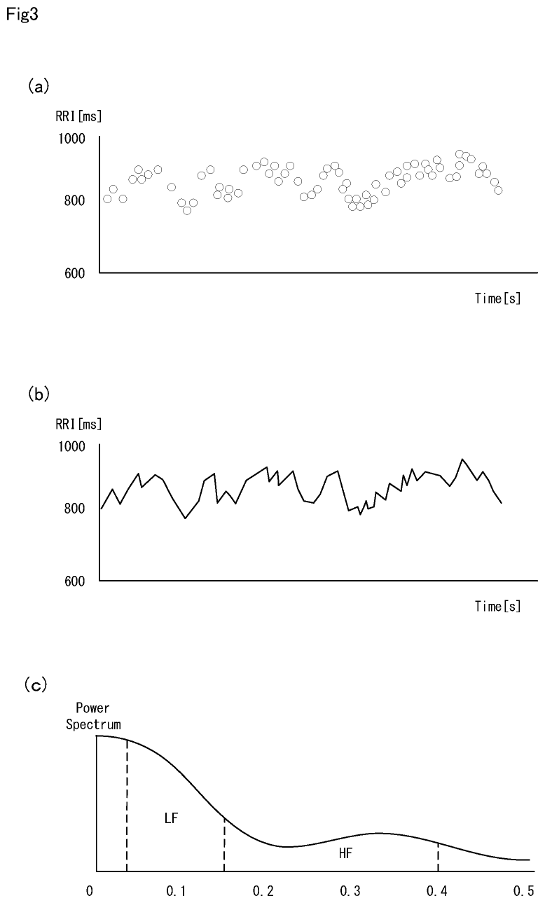

Next, the configuration and function of the fatigue level estimation apparatus according to the first example embodiment will be described in detail using FIGS. 2 and 3. FIG. 2 is a block diagram specifically showing the configuration of the fatigue level estimation apparatus according to the first example embodiment.

As shown in FIG. 2, in the first example embodiment, the fatigue level estimation apparatus 10 is connected to a terminal device 21 of the subject 20 such that data can be communicated therebetween via a wire or wirelessly. The terminal device 21 is connected to a sensor 22 for obtaining biological data. The sensor 22 is attached to the body of the subject 20. After being obtained by the sensor 22, the biological data of the subject 20 is transmitted to the terminal device 21, and thereafter, transmitted from the terminal device 21 to the fatigue level estimation apparatus 10.

Examples of the biological data include an electrocardiographic waveform, a pulse waveform, a skin potential, and a perspiration amount. In the first example embodiment, there is no limitation on the biological data as long as the biological data can be used to estimate the fatigue level. Note that, in the following description, an example will be described in which the sensor 22 outputs data indicating a heartbeat interval, such as an electrocardiographic waveform and a pulse waveform.

Also, as shown in FIG. 2, in the first example embodiment, the fatigue level estimation apparatus 10 includes a biological data obtaining unit 14, a biological data storage unit 15, and an output unit 16, in addition to the biological data extraction unit 11, the feature value calculation unit 12, and the fatigue level estimation unit 13.

When the biological data of the subject 20 is transmitted from the terminal device 21, the biological data obtaining unit 14 obtains the transmitted biological data and stores the obtained biological data in the biological data storage unit 15.

Specifically, when it is assumed that the sensor 22 outputs an electrocardiographic waveform or a pulse waveform, the terminal apparatus 21 converts the output waveform into RRI (R-R Interval) data, which is data (heartbeat fluctuation time-series data) indicating the heartbeat interval, and transmits the resultant data as the biological data. Accordingly, the biological data obtaining unit 14 stores this RRI data in the biological data storage unit 15 in association with the measurement time.

In the first example embodiment, the biological data extraction unit 11 extracts the biological data corresponding to the set time period immediately before the wake-up time of the subject, for example, the biological data (RRI data) collected 5 to 30 minutes before the subject wakes up, for example, as the biological data obtained when the subject is in a specific activity state.

Specifically, in the first example embodiment, the subject 20 inputs the time when he/she woke up into the terminal device 21 after waking up, and the terminal device 21 transmits the input time when the subject woke up to the fatigue level estimation apparatus 10. In this manner, based on the input time when the subject woke up, the biological data extraction unit 11 extracts the biological data (RRI data) corresponding to the set time period immediately before the time when the subject woke up, from the biological data storage unit 15.

When it is assumed that the biological data is data (RRI data) indicating the heartbeat interval, the feature value calculation unit 12 calculates the feature value related to heartbeat fluctuation, that is, the feature value indicating a change in the heartbeat interval for each pulse, as the feature value of the biological data. FIG. 3 is a diagram for illustrating the calculation processing of the feature value of the first example embodiment. FIG. 3(a) shows an example of original RRI data, FIG. 3(b) shows resampled RRI data, and FIG. 3(c) shows RRI data that has been subjected to frequency transformation.

Specifically, the feature value calculation unit 12 performs data interpolation on the missing portion of the RRI data shown in FIG. 3(a), using spline interpolation, for example. A substitution method can also be used for data interpolation. In the substitution method, a missing value is substituted with a sum value such as a constant and a means value. Next, as shown in FIG. 3(b), the feature value calculation unit 12 resamples the RRI data subjected to data interpolation, at 4 Hz, for example.

Next, the feature value calculation unit 12 calculates a time range feature value as the feature value, from the RRI data shown in FIG. 3(b). Also, examples of the time range feature value include the following. The feature value calculation unit 12 calculates, as the feature value, at least one of the following time range feature values.

min: RRI minimum value
max: RRI maximum value
amplitude: RRI maximum value−RRI minimum value
var: RRI variance
mrri: RRI means value
median: RRI median value
mhr: HR means value
rmssd: standard deviation of the difference between the adjacent RRIs
sdnn: RRI standard deviation (standard deviation of heartbeat interval for a given time period)
nn50: number of heartbeats when the difference between the adjacent RRIs is greater than 50 ms
pnn50: ratio of heartbeats when the difference between the adjacent RRIs is greater than 50 ms

Of the above time region feature values, sdnn is calculated using the following Expression 1. In the following Expression 1, 6 denotes the value of sdnn, and xᵢ denotes the ith heartbeat interval. Also, x̅ denotes the mean value of heartbeat interval for a given time period, while n denotes the number of heartbeat interval data pieces for a given time period. The symbol i is the number of the heartbeat interval.

$$σ = \sqrt{\frac{1}{n} {\sum }_{i = 1}^{n} {( {x}_{i} - \overline{x} )}^{2}} \qquad [ Expression 1 ]$$

The feature value calculation unit 12 may also calculate a frequency range feature value in addition to or instead of the time range feature value. In this case, as shown in FIG. 3 (c), the feature value calculation unit 12 calculates the frequency range feature value by performing Fast Fourier Transform (FFT) on the resampled RRI data to find a power spectrum density. In FIG. 3 (c), LF denotes a power spectrum in a lower frequency range (0.04 to 0.15 Hz), and HF denotes a power spectrum in a high frequency range (0.15 to 0.4 Hz).

Examples of frequency range feature values include the following. The feature value calculation unit 12 calculates at least one of the following frequency range feature values.

total_power (TP): total power of power spectrum of VLF, LF, and HF.
VLF: power spectrum in a frequency band of 0.0033 to 0.04 Hz
LF: power spectrum in a frequency band of 0.04 to 0.15 Hz
LF_nu: ratio between LF (absolute value) and (TP-vlf)
HF: power spectrum in a frequency band of 0.15 to 0.4 Hz
HF_nu: ratio between HF (absolute value) and (TP-vlf)
LF/HF: power ratio between LF and HF

In the first example embodiment, the fatigue level estimation unit 13 estimates the fatigue level of the subject by applying the feature value calculated by the feature value calculation unit 12 to a machine learning model in which the relationship between the feature value of the biological data and the fatigue level has been subjected to machine learning.

Specifically, the machine learning model for obtaining the fatigue level is constructed in advance as training data, using the feature value related to heartbeat fluctuation and the fatigue level. The fatigue level serving as the training data is calculated from answers to a questionnaire, for example.

When it is assumed that the machine learning model is F(x), the model is represented by the following Expression 2, for example. Also, in the following Expression 2, x denotes the feature value vector related to the heartbeat fluctuation, and xᵢ denotes the ith feature value (i is a natural number). The symbol y denotes the fatigue level. The symbol y serving as the training data can be obtained from answers to a questionnaire, physical capacity (jump height, maximum speed, range of motion of joints, etc.), for example. The symbol wᵢ denotes the weight of the ith feature value and is optimized through machine learning. The symbol n denotes the number of components (feature value) constituting the feature value vector n.

$$y = F ( x ) = {\sum }_{i = 1}^{n} {w}_{i} {x}_{i} \qquad [ Expression 2 ]$$

A plurality of machine learning models may also be constructed. In this case, the fatigue level estimation unit 13 combines values calculated through the machine learning models to calculate the final fatigue level. Further, the machine learning model is not limited to the linear model shown in the above Expression 2, and may also be an index model, a logarithmic model, or the like, or a combination of different models.

Also, the method for constructing the machine learning model is not particularly limited. Examples of specific methods for machine learning include linear regression, logistic regression, a support vector machine, a decision tree, a regression tree, and a neural network.

The output unit 16 transmits the fatigue level estimated by the fatigue level estimation unit 13 to the terminal device 21 of the subject 20. Accordingly, the estimated fatigue level is displayed on a screen of the terminal device 21, and thus the subject 20 can check his or her fatigue level. Also, the output unit 16 can also transmit the fatigue level estimated by the fatigue level estimation unit 13 to the terminal device of a person other than the subject 20 such as a manager of the subject, the subject's family, or a doctor. In this case, a third party other than the subject 20 can check the fatigue level of the subject 20.

[Apparatus Operation]

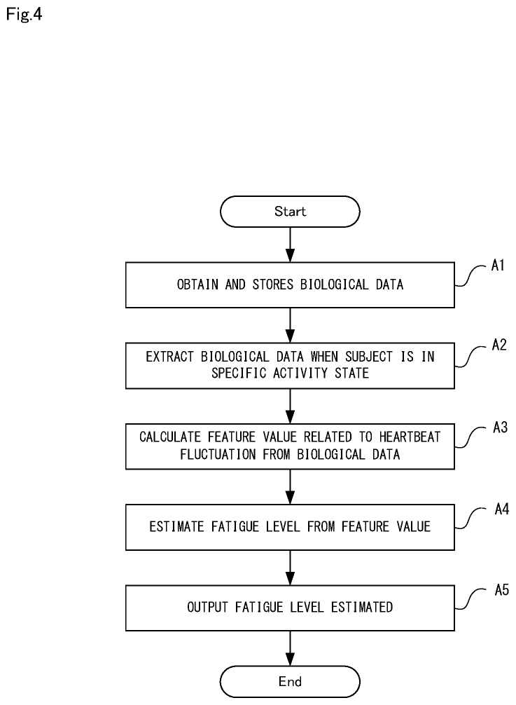

Next, operation of the fatigue level estimation apparatus 10 according to the first example embodiment will be described using FIG. 4. FIG. 4 is a flowchart showing the operation of the fatigue level estimation apparatus of the first example embodiment. In the description below, FIG. 1 to FIG. 3 are referenced as appropriate. Also, in the first example embodiment, the fatigue level estimation method is implemented by operating the fatigue level estimation apparatus 10. Accordingly, the description of the fatigue level estimation method according to the first example embodiment is replaced with the following description of the operations of the fatigue level estimation apparatus 10.

First, as shown in FIG. 4, when the biological data of the subject 20 is transmitted from the terminal device 21, the biological data obtaining unit 14 obtains the transmitted biological data, and stores the obtained biological data in the biological data storage unit 15 in time series in the order the data is transmitted (step A1).

Next, when the wake-up time of the subject 20 is transmitted from the terminal device 21, the biological data extraction unit 11 extracts the biological data corresponding to the set time period immediately before the wake-up time of the subject, as the biological data obtained when the subject is in a specific activity state (step A2).

Next, the feature value calculation unit 12 calculates a feature value of the biological data extracted in step A2 (step A3). Specifically, in step A3, the feature value calculation unit 12 calculates the feature value related to heartbeat fluctuation.

Next, the fatigue level estimation unit 13 estimates the fatigue level of the subject by applying the feature value calculated in step A3 to the machine learning model in which the relationship between the feature value of the biological data and the fatigue level has been subjected to machine learning (step A4).

Next, the output unit 16 transmits the fatigue level estimated in step A4 to the terminal device 21 of the subject 20 (step A5). In this manner, the fatigue level estimated in step A4 is displayed on the screen of the terminal device 21, and the subject 20 can check his or her fatigue level.

As described above, in the first example embodiment, since only the biological data obtained when the subject is in a specific activity state is used, the fatigue level is estimated from biological data that does not include any component that causes the fatigue level to fluctuate. Also, the feature value related to the heartbeat fluctuation is calculated from the biological data, the fatigue level is estimated from this feature value using the machine learning model, and thus the estimated fatigue level is highly reliable.

[Program]

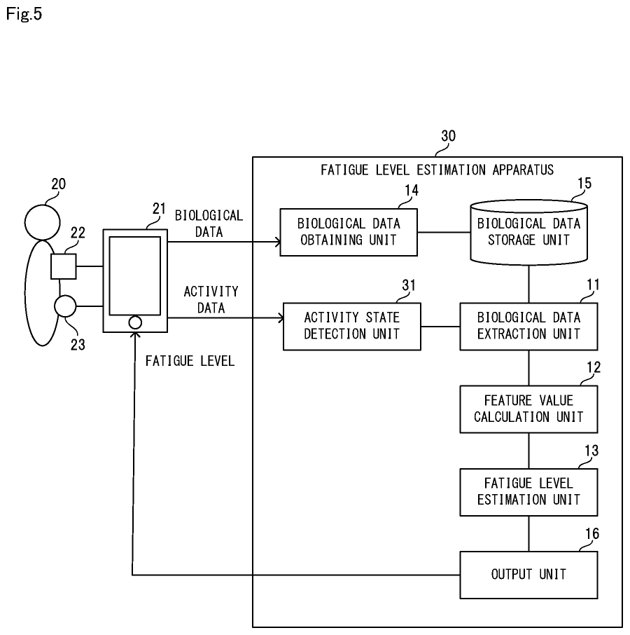

It suffices for a program in the first example embodiment to be a program that causes a computer to carry out steps A1 to A5 shown in FIG. 5. Also, by this program being installed and executed in the computer, the fatigue level estimation apparatus 10 and the fatigue level estimation method according to the first example embodiment can be realized. In this case, a processor of the computer functions and performs processing as the biological data extraction unit 11, the feature value calculation unit 12, the fatigue level estimation unit 13, the biological data obtaining unit 14, and the output unit 16.

In the first example embodiment, the biological data storage unit 15 may be realized by storing data files constituting the biological data in a storage device such as a hard disk provided in the compute. The computer includes general-purpose PC, smartphone and tablet-type terminal device.

Furthermore, the program according to the first example embodiment may be executed by a computer system constructed with a plurality of computers. In this case, for example, each computer may function as one of the biological data extraction unit 11, the feature value calculation unit 12, the fatigue level estimation unit 13, the biological data obtaining unit 14, and the output unit 16.

### Second Example Embodiment

Next, a fatigue level estimation apparatus, a fatigue level estimation method, and a program according to a second example embodiment will be described with reference to FIGS. 5 to 7.

[Apparatus Configuration]

First, the configuration of the fatigue level estimation apparatus according to the second example embodiment will be described using FIG. 5. FIG. 5 is a block diagram showing the configuration of the fatigue level estimation apparatus according to the second example embodiment.

As shown in FIG. 5, in the second example embodiment, a fatigue level estimation apparatus 30 has an activity state detection unit 31, in addition to a similar configuration to that of the fatigue level estimation apparatus 10 according to the first example embodiment. Also, in the second example embodiment, in addition to the sensor 22 for obtaining biological data, a second sensor 23 for obtaining activity data indicating the activity state of the subject is attached to the subject 20. Hereinafter, the second example embodiment will be described focusing on differences from the first example embodiment.

First, upon obtaining activity data, the second sensor 23 outputs the obtained activity data to the terminal device 21. Then, the terminal device 21 transmits the output activity data to the fatigue level estimation apparatus 30.

The activity state detection unit 31 detects the activity state of the subject 20 based on the activity data. In the second example embodiment, the biological data extraction unit 11 extracts the biological data obtained when the subject 20 is in a specific activity state, based on the result of detection by the activity state detection unit 31.

Specifically, it is assumed that a three-axis acceleration sensor is used for the second sensor 23, for example, and the second sensor 23 outputs acceleration data indicating activity by the subject as the activity data. In this case, the activity state detection unit 31 detects the fact that the activity state of the subject 20 has changed from asleep to awake, based on the activity data (acceleration data).

Then, in the second example embodiment, when it is detected that the activity state has changed from asleep to awake based on the result of detection by the activity state detection unit 31, the biological data extraction unit 11 specifies the time when the subject 20 woke up. Then, similarly to the first example embodiment, the biological data extraction unit 11 extracts the biological data corresponding to the set time period immediately before the wake-up time of the subject, as the biological data obtained when the subject is in a specific activity state.

Thereafter, similarly to the first example embodiment, the feature value calculation unit 12 calculates the feature value related to heartbeat fluctuation as the feature value of the biological data. Also, similarly to the first example embodiment, the fatigue level estimation unit 13 estimates the fatigue level of the subject by applying the calculated feature value to a machine learning model in which the relationship between the feature value of the biological data and the fatigue level has been subjected to machine learning.

[Apparatus Operation]

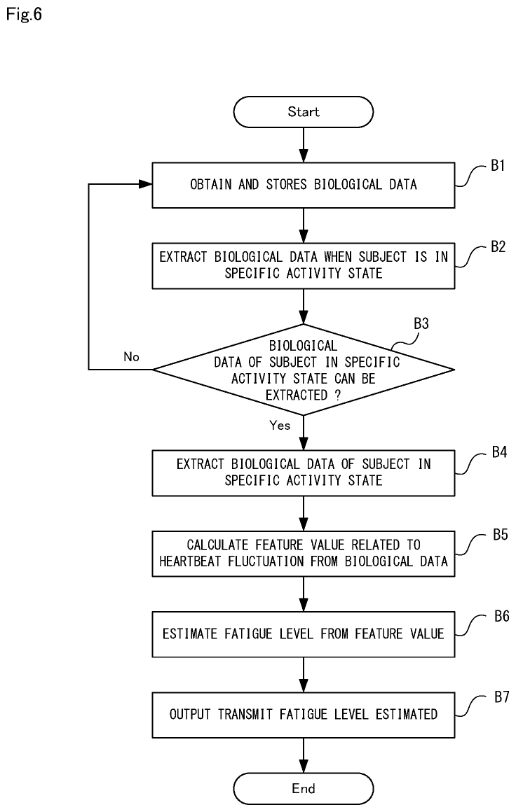

Next, operation of the fatigue level estimation apparatus 30 according to the second example embodiment will be described using FIG. 6. FIG. 6 is a flowchart showing the operation of the fatigue level estimation apparatus according to the second example embodiment. In the following description, FIG. 5 is referred to as appropriate. In the second example embodiment, the fatigue level estimation method is implemented by operating the fatigue level estimation apparatus 30. Accordingly, the description of the fatigue level estimation method according to the second example embodiment is replaced with the following description of the operations of the fatigue level estimation apparatus 30.

First, as shown in FIG. 6, when the biological data of the subject 20 is transmitted from the terminal device 21, the biological data obtaining unit 14 obtains the transmitted data, and stores the obtained biological data in the biological data storage unit 15 in time series in the order the data is transmitted (step B1).

Next, the activity state detection unit 31 detects the activity state of the subject 20 based on the activity data transmitted from the terminal device 21 (step B2).

Next, the biological data extraction unit 11 determines whether the biological data of the subject 20 in a specific activity state can be extracted, based on the result of step B2 (step B3).

Specifically, in step B2, when it is detected that the activity state of the subject 20 has changed from asleep to awake, the biological data extraction unit 11 determines “YES” in step B3. On the other hand, in step B2, if it is not detected that the activity state of the subject 20 has changed from asleep to awake, the biological data extraction unit 11 determines “NO”.

If the biological data extraction unit 11 determines “no” in step B3, step B1 is executed again. On the other hand, if the biological data extraction unit 11 determines “YES” in step B3, the biological data extraction unit 11 specifies the wake-up time of the subject 20 based on the result of detection by the activity state detection unit 31. Then, the biological data extraction unit 11 extracts the biological data corresponding to the set time period immediately before wake-up time of the subject 20 as the biological data obtained when the subject 20 is in a specific activity state (step B4).

Next, the feature value calculation unit 12 calculates the feature value of the biological data extracted in step B4 (step B5). Specifically, in step B5, the feature value calculation unit 12 calculates the feature value related to heartbeat fluctuation.

Next, the fatigue level estimation unit 13 estimates the fatigue level of the subject by applying the feature value calculated in step B5 to the machine learning model in which the relationship between the feature value of the biological data and the fatigue level has been subjected to machine learning (step B6).

Next, the output unit 16 transmits the fatigue level estimated in step B6 to the terminal device 21 of the subject 20 (step B7). In this manner, the fatigue level estimated in step B6 is displayed on the screen of the terminal device 21, and thus the subject 20 can check his or her fatigue level. In the second example embodiment as well, the output unit 16 can also transmit the fatigue level estimated by the fatigue level estimation unit 13 to the terminal device of a person other than the subject 20 such as a manager of the subject, the subject's family, or a doctor. In this case, the third party other than the subject 20 can confirm the fatigue level of the subject 20.

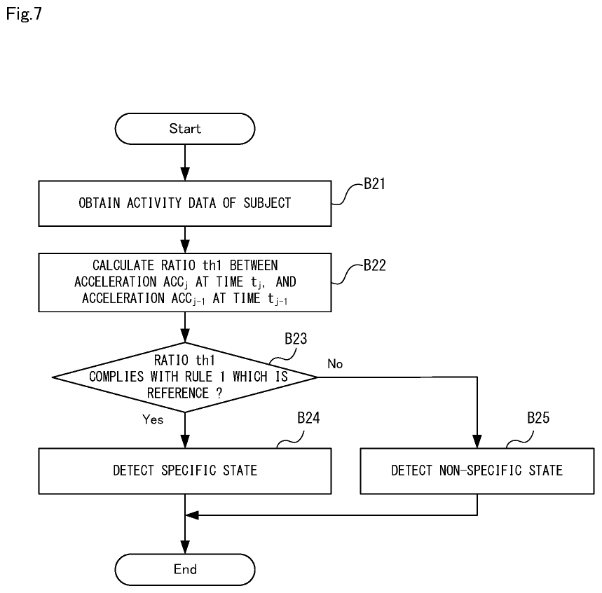

Next, step B2 shown in FIG. 6 will be described in detail using FIG. 7. FIG. 7 is a flowchart showing operations in processing for detecting the activity state performed by the fatigue level estimation apparatus in the second example embodiment.

As shown in FIG. 7, first, the activity state detection unit 31 obtains the activity data transmitted from the terminal device 21 of the subject 20, that is, the acceleration data output from the three-axis acceleration sensor, at a constant sampling rate (Step B21).

Next, the activity state detection unit 31 calculates an acceleration ACCⱼ at time tⱼ, and an acceleration ACCⱼ₋₁ at time tⱼ₋₁, and further a ratio th1 between the former and the latter (=ACCⱼ/ACCⱼ₋₁) (step B22). In step B22, the acceleration ACC is calculated using the following Expression 3, from acceleration ACCₓ in the left-right direction of the subject's body, acceleration ACC_{y} in the front-rear direction of the subject's body, and acceleration ACC_{z} in the vertical direction.

ACC=\sqrt{ACCₓ²+ACC_{y}²+ACC_{Z}²} [Expression 3]

Next, the activity state detection unit 31 determines whether the ratio th1 calculated in step B22 complies with a rule 1, which is the reference (step B23). Specifically, the activity state detection unit 31 determines whether the ratio th1 is larger than a predetermined threshold th_{wk-time}. The threshold th_{wk-time} is a threshold that is set for detecting the awake state.

As a result of determination in step B23, if the ratio th1 does not comply with the reference rule 1, the activity state detection unit 31 outputs the fact that the subject 20 is still asleep as the result of detection of the activity state (step B25).

On the other hand, as a result of the determination in step B23, if the ratio th1 complies with the reference rule 1, the activity state detection unit 31 outputs the fact that the state of the subject 20 has changed from asleep to awake at time tⱼ, as the detection result of the activity state (step B24).

As described above, in the second example embodiment, the fact that the subject is in a specific activity state is automatically detected. Accordingly, the fatigue level of the subject 20 can be estimated more accurately.

[Variation 1]

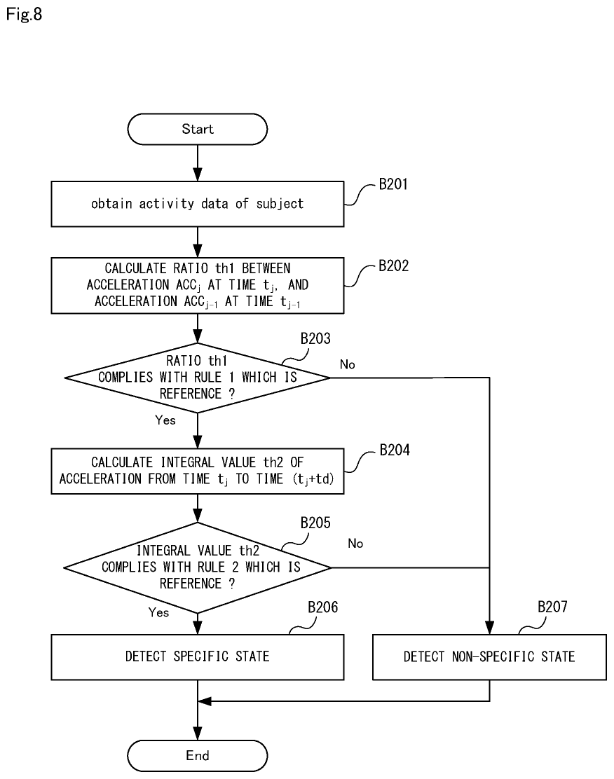

Here, a variation of the second example embodiment will be described. In variation 1, the processing in step B2 shown in FIG. 6 is different. FIG. 8 is a flowchart showing another operation in detection processing of the activity state performed by the fatigue level estimation apparatus in the second example embodiment.

As shown in FIG. 8, first, the activity state detection unit 31 obtains the activity data transmitted from the terminal device 21 of the subject 20, that is, the acceleration data output from the three-axis acceleration sensor, at a constant sampling rate (step B201). Step B201 is a step similar to step B21 shown in FIG. 7.

Next, the activity state detection unit 31 calculates the acceleration ACCⱼ at time tⱼ, and the acceleration ACCⱼ₋₁ at time tⱼ₋₁, and further the ratio th1 (=ACCⱼ/ACCⱼ₋₁) between the former and the latter (step B202). Step B202 is a step similar to step B22 shown in FIG. 7.

Next, the activity state detection unit 31 determines whether the ratio th1 calculated in step B202 complies with a rule 1, which is the reference (step B203). Step B203 is a step similar to step B23 shown in FIG. 7.

As a result of determination in step B203, if the ratio th1 does not comply with the reference rule 1, the activity state detection unit 31 outputs the fact that the subject 20 is still asleep, as the result of detection of the activity state (step B207). Step B207 is a step similar to step B25 shown in FIG. 7.

On the other hand, in variation 1, as a result of determination in step B203, if the ratio th1 complies with the reference rule 1, the activity state detection unit 31 calculates an integral value th2 of the acceleration from time tⱼ to time (tⱼ+td) (step B204).

Next, the activity state detection unit 31 determines whether the integral value th2 calculated in step B204 complies with a rule 2, which is a reference (step B205). Specifically, the activity state detection unit 31 determines whether the integral value th2 is larger than 0 and smaller than th_{wk-int} (0<th2<th_{wk-int}). The threshold th_{wk-int} is set to exclude the case where the subject 20 falls asleep again having woken up once.

As a result of determination of step B205, if the integral value th2 does not comply with the reference rule 2, the above-described step B207 is executed.

On the other hand, as a result of the determination in step B205, if the integral value th2 complies with the reference rule 2, the activity state detection unit 31 outputs the fact that the state of the subject 20 has changed from asleep to awake at time tⱼ, as the detection result of the activity state (step B206). Step B206 is a step similar to step B24 shown in FIG. 7.

In this manner, in variation 1, whether the subject 20 has fallen asleep again after waking up is also determined, which makes it possible to automatically detect the fact that the subject is in a specific activity state more accurately.

[Variation 2]

In the above-described example, the biological data corresponding to the set time period immediately before the wake-up time of the subject is extracted as the biological data obtained when the subject 20 is in a specific activity state. However, the second example embodiment is not limited to this example.

In this variation, the activity state detection unit 31 can detect the fact that the activity state has switched from REM sleep to non-REM sleep or from non-REM sleep to REM sleep. Specifically, the activity state detection unit 31 detects a switch from REM sleep to non-REM sleep or a switch from non-REM sleep to REM sleep, from an electrocardiographic waveform or a pulse waveform that is output from the sensor 22, and specifies the time when the switch occurred.

In this case, the biological data extraction unit 11 extracts the biological data corresponding to the set time period before and after the switch occurred (e.g. 5 minutes before and after the switch) as the biological data obtained when the subject is in a specific activity state.

Note that, in this variation as well, the operations of the feature value calculation unit 12, the fatigue level estimation unit 13, and the output unit 16 are similar to the above-described examples.

[Program]

It suffices for a program in the second example embodiment to be a program that causes a computer to carry out steps B1 to B7 shown in FIG. 6. Also, by this program being installed and executed in the computer, the fatigue level estimation apparatus 30 and the fatigue level estimation method according to the second example embodiment can be realized. In this case, a processor of the computer functions and performs processing as the biological data extraction unit 11, the feature value calculation unit 12, the fatigue level estimation unit 13, the biological data obtaining unit 14, the output unit 16, and the activity state detection unit 31.

In the second example embodiment, the biological data storage unit 15 may be realized by storing data files constituting the biological data in a storage device such as a hard disk provided in the compute. The computer includes general-purpose PC, smartphone and tablet-type terminal device.

Furthermore, the program according to the second example embodiment may be executed by a computer system constructed with a plurality of computers. In this case, for example, each computer may function as one of the biological data extraction unit 11, the feature value calculation unit 12, the fatigue level estimation unit 13, the biological data obtaining unit 14, the output unit 16, and the activity state detection unit 31.

[Physical Configuration]

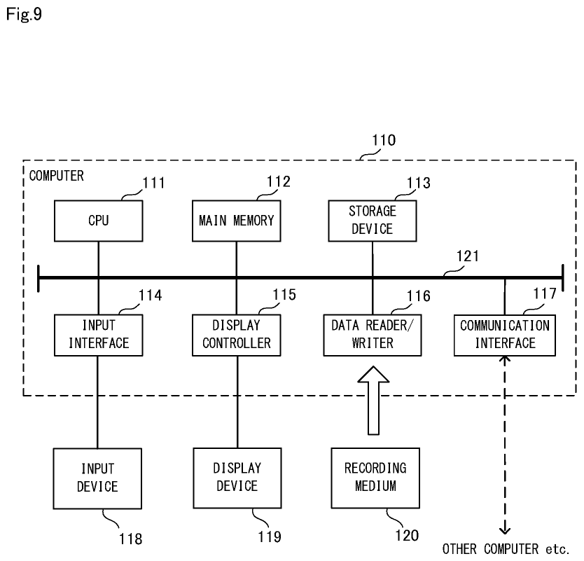

Using FIG. 9, the following describes a computer that realizes the fatigue level estimation apparatus by executing the program according to the first and second example embodiment. FIG. 9 is a block diagram illustrating an example of a computer that realizes the fatigue level estimation apparatus according to the first and second example embodiment.

As shown in FIG. 9, a computer 110 includes a CPU (Central Processing Unit) 111, a main memory 112, a storage device 113, an input interface 114, a display controller 115, a data reader/writer 116, and a communication interface 117. These components are connected in such a manner that they can perform data communication with one another via a bus 121.

The computer 110 may include a GPU (Graphics Processing Unit) or an FPGA (Field-Programmable Gate Array) in addition to the CPU 111, or in place of the CPU 111. In this case, the GPU or the FPGA can execute the programs according to the example embodiment.

The CPU 111 deploys the program according to the example embodiment, which is composed of a code group stored in the storage device 113 to the main memory 112, and carries out various types of calculation by executing the codes in a predetermined order. The main memory 112 is typically a volatile storage device, such as a DRAM (dynamic random-access memory).

Also, the program according to the example embodiment is provided in a state where it is stored in a computer-readable recording medium 120. Note that the program according to the present example embodiment may be distributed over the Internet connected via the communication interface 117.

Also, specific examples of the storage device 113 include a hard disk drive and a semiconductor storage device, such as a flash memory. The input interface 114 mediates data transmission between the CPU 111 and an input device 118, such as a keyboard and a mouse. The display controller 115 is connected to a display device 119, and controls display on the display device 119.

The data reader/writer 116 mediates data transmission between the CPU 111 and the recording medium 120, reads out the program from the recording medium 120, and writes the result of processing in the computer 110 to the recording medium 120. The communication interface 117 mediates data transmission between the CPU 111 and another computer.

Specific examples of the recording medium 120 include: a general-purpose semiconductor storage device, such as CF (CompactFlash®) and SD (Secure Digital); a magnetic recording medium, such as a flexible disk; and an optical recording medium, such as a CD-ROM (Compact Disk Read Only Memory).

Note that the fatigue level estimation apparatus according to the first and second example embodiment can also be realized by using items of hardware that respectively correspond to the components, such as a circuit, rather than the computer in which the program is installed. Furthermore, a part of the fatigue level estimation apparatus according to the first and second example embodiment may be realized by the program, and the remaining part of the fatigue level estimation apparatus may be realized by hardware.

A part or an entirety of the above-described example embodiment can be represented by (Supplementary Note 1) to (Supplementary Note 18) described below but is not limited to the description below.

(Supplementary Note 1)

A fatigue level estimation apparatus comprising:
a biological data extraction unit that extracts biological data obtained when a subject is in a specific activity state, from biological data obtained from the subject;
a feature value calculation unit that calculates a feature value of the biological data, based on the extracted biological data obtained when the subject is in a specific activity state; and
a fatigue level estimation unit that estimates a fatigue level indicating a level of fatigue of the subject, based on the calculated feature value.

(Supplementary Note 2)

The fatigue level estimation apparatus according to claim 1, further comprising
an activity state detection unit that detects an activity state of the subject,
wherein the biological data extraction unit extracts the biological data obtained when the subject is in a specific activity state, from the obtained biological data, based on a result of detection by the activity state detection unit.

(Supplementary Note 3)

The fatigue level estimation apparatus according to claim 2,
wherein, when the activity state detection unit detects that an activity state of the subject has changed from asleep to awake,
the biological data extraction unit extracts biological data corresponding to a set time period immediately before a wake-up time of the subject, as the biological data obtained when the subject is in a specific activity state.

(Supplementary Note 4)

The fatigue level estimation apparatus according to claim 2,
wherein, when the activity state detection unit detects a fact that the activity state has switched from REM sleep to non-REM sleep, or the activity state has switched from non-REM sleep to REM sleep,
the biological data extraction unit extracts the biological data corresponding to a set time immediately before and after a time when the activity state switched, as the biological data obtained when the subject is in a specific activity state.

(Supplementary Note 5)

The fatigue level estimation apparatus according to any one of claims 1 to 4,
wherein, when the biological data is data indicating a heartbeat interval,
the feature value calculation unit calculates a feature value indicating a change in a pulse interval for each pulse, as the feature value.

(Supplementary Note 6)

The fatigue level estimation apparatus according to any one of claims 1 to 5,
wherein the fatigue level estimation unit estimates the fatigue level of the subject by applying the calculated feature value to a machine learning model in which a relationship between the feature value of the biological data and the fatigue level has been subjected to machine learning.

(Supplementary Note 7)

A fatigue level estimation method comprising:
a biological data extraction step of extracting biological data obtained when a subject is in a specific activity state, from biological data obtained from a subject;
a feature value calculation step of calculating a feature value of the biological data, based on the extracted biological data obtained when the subject is in a specific activity state; and
a fatigue level estimation step of estimating a fatigue level indicating a level of fatigue of the subject, based on the calculated feature value.

(Supplementary Note 8)

The fatigue level estimation method according to claim 7, further comprising:
detecting an activity state of the subject,
wherein, in the biological data extraction step, the biological data obtained when the subject is in a specific activity state is extracted, from the obtained biological data, based on a result of detection by the activity state detection.

(Supplementary Note 9)

The fatigue level estimation method according to claim 8,
wherein, in the activity state detection step, when it is detected that an activity state of the subject has changed from asleep to awake,
in the biological data extraction step, biological data corresponding to a set time period immediately before a wake-up time of the subject is extracted, as the biological data obtained when the subject is in a specific activity state.

(Supplementary Note 10)

The fatigue level estimation method according to claim 8,
wherein, in the activity state detection step, when it is detected that the activity state has switched from REM sleep to non-REM sleep, or the activity state has switched from non-REM sleep to REM sleep,
in the biological data extraction step, extracting the biological data corresponding to a set time period immediately before and after a time when the activity state switched, as the biological data obtained when the subject is in a specific activity state.

(Supplementary Note 11)

The fatigue level estimation method according to any one of claims 7 to 10,
wherein, when the biological data is data indicating a heartbeat interval,
in the feature value calculation step, a feature value indicating a change in a pulse interval for each pulse is calculated, as the feature value.

(Supplementary Note 12)

The fatigue level estimation method according to any one of claims 7 to 11,
wherein, in the fatigue level estimation step, a fatigue level of the subject is estimated by applying the calculated feature value to a machine learning model in which a relationship between the feature value of the biological data and the fatigue level has been subjected to machine learning.

(Supplementary Note 13)

A computer-readable recording medium on which a program is recorded, the program comprising instructions that cause a computer to carry out:
a biological data extraction step of extracting biological data obtained when a subject is in a specific activity state, from biological data obtained from a subject;
a feature value calculation step of calculating a feature value of the biological data, based on the extracted biological data obtained when the subject is in a specific activity state; and
a fatigue level estimation step of estimating a fatigue level indicating a level of fatigue of the subject, based on the calculated feature value.

(Supplementary Note 14)

The computer-readable recording medium according to claim 13, wherein the program causes the computer to further carry out:
detecting an activity state of the subject,
wherein, in the biological data extraction step, the biological data obtained when the subject is in a specific activity state is extracted, based on a result of detection by the activity state detection.

(Supplementary Note 15)

The computer-readable recording medium according to claim 14,
wherein, in the activity state detection step, when it is detected that an activity state of the subject has changed from asleep to awake,
in the biological data extraction step, biological data corresponding to a set time period immediately before a wake-up time of the subject is extracted, as the biological data obtained when the subject is in a specific activity state.

(Supplementary Note 16)

The computer-readable recording medium according to claim 14,
wherein, in the activity state detection step, when it is detected that the activity state has switched from REM sleep to non-REM sleep, or the activity state has switched from non-REM sleep to REM sleep,
in the biological data extraction step, extracting the biological data corresponding to the set time period immediately before and after a time when the activity state switched, as the biological data obtained when the subject is in a specific activity state.

(Supplementary Note 17)

The computer-readable recording medium according to any one of claims 13 to 16,
wherein, when the biological data is data indicating a heartbeat interval, in the feature value calculation step, a feature value indicating a change in a pulse interval for each pulse is calculated, as the feature value.

(Supplementary Note 18)

The computer-readable recording medium according to any one of claims 13 to 17,
wherein, in the fatigue level estimation step, a fatigue level of the subject is estimated by applying the calculated feature value to a machine learning model in which a relationship between the feature value of the biological data and the fatigue level has been subjected to machine learning.

Although the invention of the present application has been described above with reference to the example embodiment, the invention of the present application is not limited to the above-described example embodiment. Various changes that can be understood by a person skilled in the art within the scope of the invention of the present application can be made to the configuration and the details of the invention of the present application.

### INDUSTRIAL APPLICABILITY

As described above, according to the invention, it is possible to estimate the fatigue level by obtaining biological data that does not include any component that causes the fatigue level to fluctuate. The invention is useful, for example, in health management systems, personnel management systems, and the like.

### REFERENCE SIGNS LIST

10 Fatigue level estimation apparatus
11 Biological data extraction unit
12 Feature value calculation unit
13 Fatigue level estimation unit
14 Biological data obtaining unit
15 Biological data storage unit
16 Output unit
20 Subject
21 Terminal device
22 Sensor
23 Second sensor
30 Fatigue level estimation apparatus
110 Computer
111 CPU
112 Main memory
113 Storage device
114 Input interface
115 Display controller
116 Data reader/writer
117 Communication interface
118 Input device
119 Display device
120 Recording medium
121 Bus

What is claimed is:

1. A fatigue level estimation apparatus comprising:
at least one memory storing instructions; and
at least one processor configured to execute the instructions to:
extract biological data obtained when a subject is in a specific activity state, from biological data obtained from the subject;
calculate a feature value of the biological data, based on the extracted biological data obtained when the subject is in a specific activity state; and
estimate a fatigue level indicating a level of fatigue of the subject, based on the calculated feature value.

2. The fatigue level estimation apparatus according to claim 1, further,
further at least one processor configured to execute the instructions to:
detect an activity state of the subject,
extract the biological data obtained when the subject is in a specific activity state, from the obtained biological data, based on a result of detection by the activity state detection means.

3. The fatigue level estimation apparatus according to claim 2,
further at least one processor configured to execute the instructions to:
when it is detected that an activity state of the subject has changed from asleep to awake,
extract biological data corresponding to a set time period immediately before a wake-up time of the subject, as the biological data obtained when the subject is in a specific activity state.

4. The fatigue level estimation apparatus according to claim 2,
further at least one processor configured to execute the instructions to:
when it is detected that the activity state has switched from REM sleep to non-REM sleep, or the activity state has switched from non-REM sleep to REM sleep,
extract biological data corresponding to a set time immediately before and after a time when the activity state switched, as the biological data obtained when the subject is in a specific activity state.

5. The fatigue level estimation apparatus according to claim 1,
further at least one processor configured to execute the instructions to:
when the biological data is data indicating a heartbeat interval,
calculate a feature value indicating a change in a pulse interval for each pulse, as the feature value.

6. The fatigue level estimation apparatus according to claim 1,
further at least one processor configured to execute the instructions to:
estimate the fatigue level of the subject by applying the calculated feature value to a machine learning model in which a relationship between the feature value of the biological data and the fatigue level has been subjected to machine learning.

7. A fatigue level estimation method comprising:
extracting biological data obtained when a subject is in a specific activity state, from biological data obtained from a subject;
calculating a feature value of the biological data, based on the extracted biological data obtained when the subject is in a specific activity state; and
estimating a fatigue level indicating a level of fatigue of the subject, based on the calculated feature value.

8. The fatigue level estimation method according to claim 7, further comprising:
detecting an activity state of the subject,
wherein, in the biological data extraction, the biological data obtained when the subject is in a specific activity state is extracted, from the obtained biological data, based on a result of detection by the activity state detection.

9. The fatigue level estimation method according to claim 8,
wherein, in the activity state detection, when it is detected that an activity state of the subject has changed from asleep to awake,
in the biological data extraction, biological data corresponding to a set time period immediately before a wake-up time of the subject is extracted, as the biological data obtained when the subject is in a specific activity state.

10. The fatigue level estimation method according to claim 8,
wherein, in the activity state detection, when it is detected that the activity state has switched from REM sleep to non-REM sleep, or the activity state has switched from non-REM sleep to REM sleep,
in the biological data extraction, extracting the biological data corresponding to a set time period immediately before and after a time when the activity state switched, as the biological data obtained when the subject is in a specific activity state.

11. The fatigue level estimation method according to claim 7,
wherein, when the biological data is data indicating a heartbeat interval,
in the feature value calculation, a feature value indicating a change in a pulse interval for each pulse is calculated, as the feature value.

12. The fatigue level estimation method according to claim 7,
wherein, in the fatigue level estimation, a fatigue level of the subject is estimated by applying the calculated feature value to a machine learning model in which a relationship between the feature value of the biological data and the fatigue level has been subjected to machine learning.

13. A non-transitory computer-readable recording medium on which a program is recorded, the program comprising instructions that cause a computer to carry out:
extracting biological data obtained when a subject is in a specific activity state from biological data obtained from the subject;
calculating a feature value of the biological data based on the extracted biological data obtained when the subject is in a specific activity state; and
estimating a fatigue level indicating a level of fatigue of the subject, based on the calculated feature value.

14. The non-transitory computer-readable recording medium according to claim 13, wherein the program causes the computer to further carry out
detecting an activity state of the subject,
wherein, in the biological data extraction, the biological data obtained when the subject is in a specific activity state is extracted, based on a result of detection by the activity state detection.

15. The non-transitory computer-readable recording medium according to claim 14,
wherein, in the activity state detection, when it is detected that an activity state of the subject has changed from asleep to awake,
in the biological data extraction, biological data corresponding to a set time period immediately before a wake-up time of the subject is extracted, as the biological data obtained when the subject is in a specific activity state.

16. The non-transitory computer-readable recording medium according to claim 14,
wherein, in the activity state detection, when it is detected that the activity state has switched from REM sleep to non-REM sleep, or the activity state has switched from non-REM sleep to REM sleep,
in the biological data extraction, extracting the biological data corresponding to the set time period immediately before and after a time when the activity state switched, as the biological data obtained when the subject is in a specific activity state.

17. The non-transitory computer-readable recording medium according to claim 13,
wherein, when the biological data is data indicating a heartbeat interval,
in the feature value calculation, a feature value indicating a change in a pulse interval for each pulse is calculated, as the feature value.

18. The non-transitory computer-readable recording medium according to claim 13,
wherein, in the fatigue level estimation, a fatigue level of the subject is estimated by applying the calculated feature value to a machine learning model in which a relationship between the feature value of the biological data and the fatigue level has been subjected to machine learning.
